(19) United States
(12) Patent Application Publication
(10) Pub. No.: US 2018/0271636 A1
(43) Pub. Date: Sep. 27, 2018

(54) IVC FILTER RETRIEVAL SYSTEM SHEATH IMPROVEMENTS

(71) Applicant: AVANTEC VASCULAR CORPORATION, Sunnyvale, CA (US)

(72) Inventors: Nicholas deBeer, Sunnyvale, CA (US); Teresa Ruvalcaba, Sunnyvale, CA (US); Karl Halden, Sunnyvale, CA (US); Frank Becking, Sunnyvale, CA (US)

(21) Appl. No.: 15/989,845

(22) Filed: May 25, 2018

Related U.S. Application Data
(63) Continuation of application No. PCT/US16/65815, filed on Dec. 9, 2016.
(60) Provisional application No. 62/265,883, filed on Dec. 10, 2015.

(51) Int. Cl. A61F 2/01

(52) U.S. Cl. CPC A61F 2/01; A61B 2017/22035; A61F 2002/011

(57) ABSTRACT

Funnel-trap type delivery and/or retrieval devices for Inferior Vena Cava (IVC) filters or other medical implants are described with associated sheaths. The sheaths are used in progressive recapture and/or embolic protection methods or in association with such features.

### CROSS-REFERENCE TO RELATED APPLICATIONS

This application is a continuation of PCT Application No. PCT/US2016/065815, filed Dec. 9, 2016, which claims priority to and the benefit of U.S. Provisional Patent Application No. 62/265,883, filed Dec. 10, 2015, both of which are incorporated by reference herein in their entireties for all purposes.

### FIELD

The embodiments described herein relate to endovascular temporary Inferior Vena Cava (IVC) filter or other implant retrieval devices or system and methods.

### BACKGROUND

Temporary IVC filters are placed much like permanent filters, but are designed so that they may be retrieved in a separate endovascular procedure, generally from a femoral vein or internal jugular vein approach. Most of the currently available temporary filters include a hook-like feature with which they can be captured and received within a catheter or sheath for removal by employing a gooseneck snare or a multi-loop snare.

While retrieval is a simple procedure in principle, difficulty is often encountered capturing a filter's hook with the snare loop(s). Such difficulty is compounded when the filter is tilted or off-kilter in placement. Several filters are designed to avoid such orientation. However, the problem remains common because the device is not anchored into the IVC in a stable fashion. Constant blood flow in addition to blood clots can disorient the filter within the IVC making recapture difficult.

Accordingly, there exists a need for a filter retrieval system with improved ease of use and/or less susceptibility to problems of filter orientation.

### SUMMARY

PCT/US2014/042343 discloses a highly advantageous retrieval system comprising a folded-back braid element mounted as an extension to a pusher shaft. Various sheaths are described herein that are designed for coordinated use in covering the folded-back extension to secure a/the proximal end of a medical device (often and IVC filter) for recapture. In some embodiments, the sheaths include features to improve the recapture interface. In other sheath embodiments, features are provided for embolic protection in association with the recapture methods. Sheaths incorporating both types or sets of features are also contemplated.

Regarding the improved recapture interface features, these are such that the sheath provides for improved locking or lock-up of a proximal enlargement of the filter with the retrieval device. In some embodiments, such function is accomplished with a funnel-shape or tapered form to the sheath. In another embodiment, improved locking function is accomplished with an internal balloon. Further alternatives contemplated include internal features selected from internal fins or strips of material and leaf spring members. Yet another approach may employ a radically expandable stent-type architecture, optionally embedded in the sheath wall.

Regarding features provided for embolic protection, a funnel-shaped embolic protector may be provided. The protector can be made of braid. Optionally, the braid comprises superelastic Nitinol material heatset in the desired shape.

Any such embolic protector can be used in connection with a secondary sheath for actuation and/or stowage. Alternatively, a funnel-shaped embolic protector may be affixed to or integrated with a/the pusher shaft of the recapture and retrieval device—alone or with the various sheath embodiments.

The subject delivery and/or retrieval devices, components, kits in which they are included (with and without assembly), methods of use and manufacture (including assembly of the constituent components in vivo or ex vivo) are all included within the scope of the present disclosure. Some aspects of the same are described above, more detailed discussion is presented in connection with the figures below.

### BRIEF DESCRIPTION OF THE DRAWINGS

The figures diagrammatically illustrate inventive embodiments. Variations other than those shown in the figures are contemplated as described in a broader sense per the Summary above, as generically claimed, or otherwise.

FIGS. 1A and 1B picture IVC filter variations as may be used in the present system.

FIG. 2 is a partially cross-sectioned side view of a retrieval system with a sheath including an embolic protector.

FIG. 3 is a side-sectional view of a converted preform (i.e., a finally shaped funnel section of the retrieval system) after heatsetting.

FIG. 4 is a side-sectional view of a retrieval system with a compression sheath.

FIGS. 5A-5F are side-sectional views illustrating various compression sheath options and features.

FIG. 6 is a flowchart detailing methods of system use.

### DETAILED DESCRIPTION

Various exemplary embodiments are described below. Reference is made to these examples in a non-limiting sense, as it should be noted that they are provided to illustrate more broadly applicable aspects of the devices, systems and methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present invention. All such modifications are intended to be within the scope of the claims made herein.

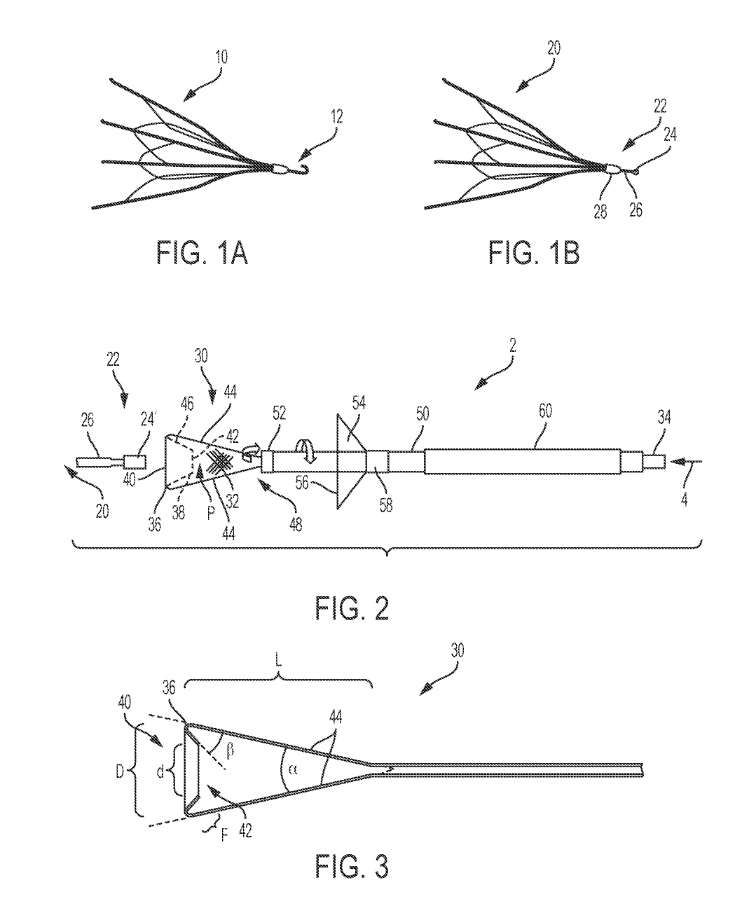

FIG. 1A shows a GÜNTHER TULIP (Cook Medical, Inc.) temporary IVC filter 10 with a hook 12 end interface for retrieval. As shown in FIG. 1B for an IVC filter 20, the hook may be modified or substituted for a nubbin-type interface 22. The nubbin may comprise a laser-formed or solder-formed protuberance or bump 24 on an extension 26 from a hub 28. Alternatively, as shown in FIG. 2, a/the filter retrieval interface 22 may comprise a band 24′ (e.g., a Pt marker band) mounted (e.g., by swaging, welding, gluing, etc.) on a/the extension 26. However, the enlargement is created (e.g., as a hook or a bump), the funnel-trap structures described below are adapted to secure that feature for IVC filter retrieval.

FIG. 2 provides an overview of a retrieval system 2. A funnel-trap structure 30 is shown made of heatset braid material 32. The construction provides a flexible distal extension to an elongate shaft 34. The shaft is received within an elongate sleeve 50. The sleeve may be a commercially available catheter or a custom part of the overall system 2) and may include a distal radiopaque marker band 52. Alone (i.e., without the sleeve) retrieval device 4 may include the funnel trap structure 30, shaft 34, a shaping wire received therein (not shown) and associated features such as support member “fingers” as referenced further below.

As a custom part, the sleeve or sheath 50 may have a secondary funnel 54 mounted thereto. The purpose of such a body is to serve for embolic protection upon deployment. Opened (ideally, though not necessarily) to the full diameter of the IVC, it is configured with a braid density (or other construction porosity) to catch any thrombus, emboli, including micro-emboli that may be dislodged from the filter 10/20 during a recapture procedure.

The secondary funnel member 54 may be made somewhat as described below and/or in the referenced patent applications below. In one example, the funnel comprises a folded-back heatset section of braid with a distal fold 56 and proximal shaft 58 including inner and outer two braid layers. These layers may be fused to the sleeve 50 using conventional catheter construction techniques.

When sheath 50 includes a secondary funnel 54, a secondary sheath 60 may be included in the system for collapsing funnel 54 and stowing it for tracking with the vasculature and uncovering it allowing expansion at a target site or location. As with sheath 50, sheath 60 may be a commercially available catheter (such as the access catheter or sheath used to gain vascular access) or a custom part of the overall system 100 and may include a distal radiopaque marker band (not shown).

The so-called secondary sheath 60 may alternatively (or additionally) be used to cover and collapse the legs of a filter once the retrieval interface 22 has been captured using sheath 50. In other words, once sheath 50 is advanced over the funnel trap section 30 capturing the retrieval interface (be it a hook 12 or nubbin 24) within a pocket (P), then sheath 60 can be advanced over the rest of the implant (optionally, without also advancing sheath 50 any further).

The braid of each funnel (i.e., funnel trap 30 and/or embolic protection funnel 54) may comprise Nitinol (preferably that is superelastic at body temperature), CoCr, Stainless Steel or another biocompatible material. The braid advantageously incorporates between 72 and 192 filament “ends” in a 1-over-1, 1-over-2, 2-over-2 or other pattern. With (superelastic) Nitinol, the wire is advantageously between about 0.001 and about 0.003 inches in diameter.

In which case, a supple and relatively “smooth” matrix surface is provided from which to construct the funnel trap architecture shown and described. The value of such a surface in the funnel trap is in its atraumatic aspect and/or ability to help guide in IVC filter (re)capture interface into position for capture even if it is oriented off-angle.

While still offering an atraumatic interface with the IVC at its fold 56, braid in the embolic protection funnel 54 may be generally in the lower range of end count noted above as this element is still meant to pass blood while capturing emboli. Indeed, braid incorporating fewer than 72 filaments may be used in constructing this body. Likewise, other wire size and/or end count in a braid or other construction funnel-trap 30 are possible as well.

To further assist with recapture, the funnel trap structure 30 may be selectably directable. As indicated by the arrows in FIG. 2, the material from which it is made can be heatset or otherwise configured to provide a bias in an angular direction. The angle of deployment may be selectable or fully straightened by relative position of a core member or obturator (not shown) or by a sleeve or catheter sheath. Further positioning may be achieved by rotating the device. Alternatively, a curved, “L” or “J” shaped wire may be received within a lumen of shaft 34 that can be passed up to and/or through to the inside of the funnel trap structure. Made of superelastic Nitinol (or other) wire, this member can be used to selectively shape or direct the device end.

Other device articulation options for selecting the angular orientation of the funnel-trap portion of the device are possible as well. Any of a variety of steerable or directable catheter-type technologies (reliant on pull-wires or otherwise) can be incorporated in shaft 34 for such purposes. Examples include the mechanisms described in U.S. Pat. Nos. 4,723,936; 4,960,411; 6,251,092 and 8,273,073 each incorporated herein by reference in its entirety for such description.

The subject funnel trap and/or secondary funnel may be generally frusto-conical in shape (as shown) or otherwise configured. With an outer conical shape (i.e., with a triangular or trapezoidal shape in cross section as illustrated) the funnel trap structure is highly supportive for any necessary or desirable tissue discretion that might need to occur to free an emplaced filter. Moreover, such a shape provides a flexible “waist” section 48 for the directable feature(s) noted above. Nevertheless, the so-called funnel shaped device may be bowed outward along its sides or otherwise configured.

As illustrated in FIG. 2, a distal rim opening 40 of the funnel trap structure 30 is larger than its more proximal rim opening 42 to operate in guiding filter engagement feature(s) or enlargement 24/24′ into a pocket (P) where the enlargement is captured and subsequently locked upon advancing sleeve 50. Such a pocket is formed between braid walls 44 and bend 38, optionally serving as an abutment feature with an edge or shoulder of nubbin/bump 24/24′.

To ensure capture, the sleeve 50 may be advanced fully over trap 30 before withdrawal into a separate catheter. In other words, advancing sleeve 50 over funnel section 30 “closes the trap” and securely captures the implant to be retrieved. Otherwise, the sleeve may be a catheter.

Notably, system 2 may be used identically when capturing a filter 10 with a typical hook end 12. However, the additional bulk/lateral extension of the hook may necessitate use of a relatively larger sleeve or catheter 50 and/or benefit from the use of the progressive-compression type sleeve components detailed below. In any case, system use may be visualized fluoroscopically by a physician by way of marker features 24/24′ and 52 and/or others as may be conveniently provided.

In the various system architectures, the catheter/pusher shaft and/or sleeve may comprise a simple extrusion (e.g., PTFE, FEP, PEEK, PI, etc.) or may be constructed using conventional catheter construction techniques and include a liner, braid support and/or outer jacket (not shown), metal hypotube, etc. Further, the filter frame may be constructed using conventional laser cutting and electropolishing techniques and/or be otherwise constructed. In embodiments intended for tracking through a guide/delivery catheter without an incorporated sheath, a loading sheath may be employed. Such a loading sheath may be splittable. Other typical percutaneous access instruments (such as wires, etc.), valves and other hardware may also be employed in connection with the invention embodiments.

The funnel-trap structure 30 may be produced as a subassembly and attached to the catheter/pusher shaft. PCT Publication No. WO 2014/201380 and U.S. patent application Ser. No. 14/569,500, now U.S. Patent Publication No. 2015/0105819, each incorporated by reference in its entirety, detail optional steps in the manufacture of a pre-form for constructing the funnel-trap portion 30 as shown if FIG. 3 to be used in the final device.

For IVC filter retrieval. the funnel-trap portion 30 shown may have a diameter (D) from about 5 mm to about 2.5 mm, or more preferably about 15 to about 20 mm (i.e., sized in a range to work within average size human IVCs where such vessels are reported as having a mean diameter of 20 mm within a range of 13 to 30 mm). A length (L) may range from about 10 mm to about 30 mm. An overall cone angle (α) between braid walls 44 may be between about 30 and about 90 degrees. An angle (β) of bend 36 between braid wall 44 and flap 46 may be between about 0 and about 60 degrees and flap length (F) may be between about 1 and about 10 mm in length. Overall, a funnel trap opening diameter (d) may be between about 5 and about 95 percent of diameter (D) depending on the selected combination of the noted variables (i.e., d, D, L, F, α and β). At the lower end of this range, the inner “opening” may be substantially closed such that it must be pushed-open to receive the proximal engagement feature(s) of the implant during retrieval. At the higher end of the range, the flap may lie completely along or in-line with the outer layer(s) of the device. The opening 40 of the funnel trap may be set at 90 degrees relative to a device axis as shown. Otherwise, it may be angled or have a more complex shape as described in connection with FIGS. 9-13 in the above-referenced U.S. patent application Ser. No. 14/569,500 incorporated herein by reference.

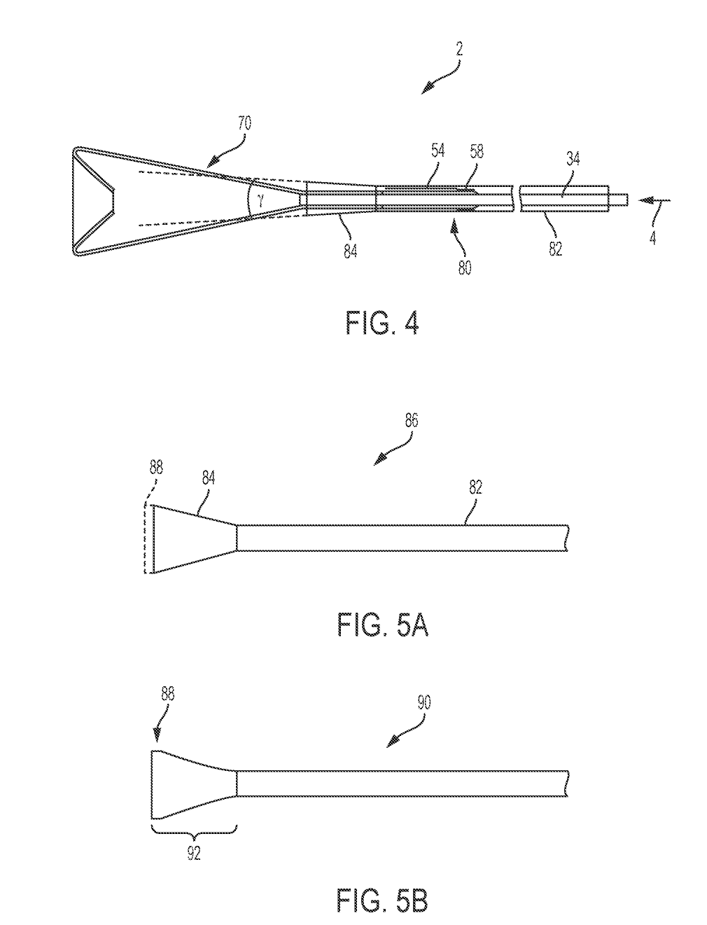

As further described in U.S. Provisional Patent Application No. 62/091,433 and U.S. patent application Ser. No. 14/965,500, now U.S. Publication No. 2016/0166372, each incorporated by reference herein in its entirety, embodiments hereof may include a support member(s) set within the funnel trap section of the device. As shown in FIG. 4, a support member 70 may be interposed between braid layers.

The focus here, however, is the illustrated form of sheath 80 and the option of including an embolic protection funnel member 54 underlying the sheath. If such a funnel is provided, its shaft section 58 can be bonded to device shaft 34 in the same fashion (even in the same procedure) as bonding the funnel section 30 thereto.

As to the sheath 80, it includes a proximal (typically) cylindrical section 82 and a flared distal section 84. The flared section may be conical and produced using conventional “tip-flaring” techniques when the sheath comprises a thermoplastic polymer. The sheath may be braid-reinforced or a simple extrusion.

The low angle employed in the tapered section is among the distinguishing features of the sheath. For example, the included angle (γ) of the conical shape may be between 5 and about 15 degrees. As a subset, the angle may be between about 7 and 12 degrees. In either case, the purpose of the taper so-defined is to progressively compress the capture interface securing the interface section 12 or 22 of the filter within the funnel-trap pocket (P). With the progressive mode to compression in this embodiment, the sheath is advanced until tight during use.

In some cases, the sheath can be advanced such that the taper region 84 passes the capture zone or region (as defined by pocket, P, shown in FIGS. 2 and 4) so that the sheath proximal cylindrical section 82 overlays the zone in use. That will typically be the case when capturing filters with relatively smaller proximal ends or apices. Filters with a relatively larger end will typically be captured with the end (i.e., interface portion 12 or 22 in the examples shown in FIGS. 1A and 1B) overlaid with the tapered section 84.

As such, the tapered sheath can provide a measure of sizing flexibility to sheath 50 for capturing a variety of filters. Alternatively, the taper can provide accommodation to variations between filters and/or simply the differences of filter position relative to the funnel trap. For example, when the hub 28 of a filter 20 overlaps with proximal bend 38 the stack-up can be greater than otherwise experienced. With the progressive compression offered by the tapered sheath 80, however, such differences in component dimensional stack-up is not problematic.

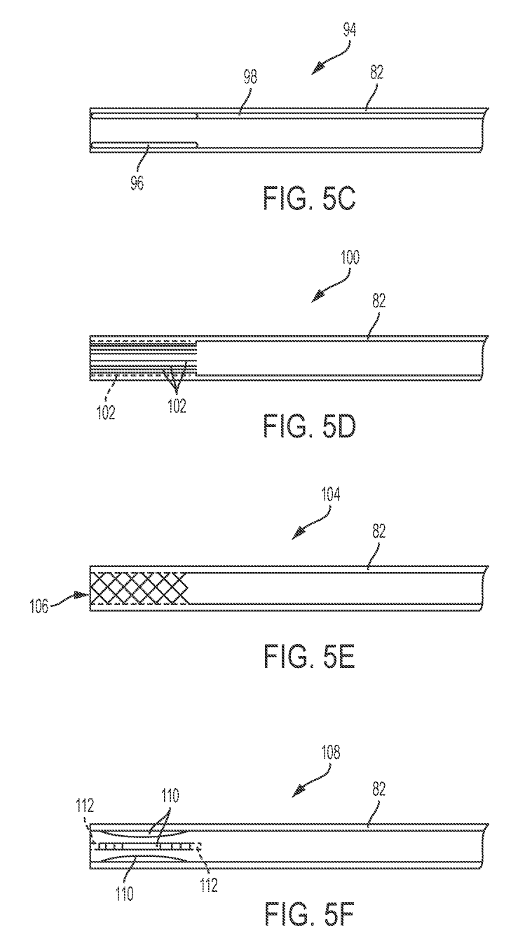

Operating according to similar principles, sleeve 86 shown in FIG. 5A may include a cylindrical lead-in section or extension 88 adjacent to the conical compression section. Such an extension may carry a radiopaque marker (not shown) for improved visualization. Alternatively, this section may be thinned-out relative to the remainder of the body to better allow ovalization to accommodate off-center filter recapture and/or retrieval device positioning.

The sheath embodiment 90 in FIG. 5B includes such an extension, as well as a curvilinear taper (vs. a simple conical) section 92. Employing a curvilinear shape (e.g., as in an “S” turn or portion thereof) may be useful in easing friction at transition areas within the sheath.

In FIG. 5C illustrates another sheath embodiment in cross section. This sheath 94 includes a hollow cylindrical body 82 carrying an internal balloon 96 fed by an inflation lumen 98. A single inflation lumen is shown. Otherwise, the inflation “lumen” may be torodial as with the balloon. In which case, the entire interior of the catheter may be regarded as a balloon. Otherwise, the proximal or feed section of the lumen may be reinforced so that it does not expand like the intended distal balloon section 96.

In any case, sheath 94 is used like those above in covering the recapture zone of the filter/implant interface. Then, the region is progressively compressed upon balloon inflation. Such compression may altogether lock-up the various components, including the sheath 94 to the underlying retrieval funnel 30 and/or shaft 34.

Sheath 100 in FIG. 5D includes internal fins 102. These may number anywhere between three and eight or more. The intent is for the strips of material (i.e., fins) to interact with the funnel section braid 32 pushing it inwardly in local areas. Typically, though not necessarily, the fins will be evenly spaced around the inner circumference of a/the cylindrical sheath body 82.

They may be formed integrally with the sheath body. Alternatively, they may be fused therewith, especially in a termoplastic construction, using conventional catheter manufacture techniques. Otherwise they may be welded (e.g., using ultrasonics) or otherwise bonded in place. Individual fins may be so-placed. Or they may take be provided in the form of an insert produced as a separate extrusion that is secure in place by fusing, welding, bonding, etc.

If somewhat rigid, the fins can provide highly localized regions of increased push or strain on the braid providing implant interface. If more compliant, the fins may perform more as integrated spring strips or sections. In higher numbers, the force the fins apply will be more uniform. In lower numbers, the force more localized. In any case, the intent is to provide a more secure interface of the funnel with the implant portion to be secured within pocket P.

With sheath 104 shown in FIG. 5E, a uniformly spaced or surrounding spring member in the form of a stent body 106 is included in the design. The stent or stent like body 106 may be embedded in the wall 82 of the sheath. Alternatively, it may be carried on the outside.

Optionally laser cut from tubular Nitinol stock (hypotube), electropolished and heatset (typically) in an expanded condition, the stent section or member 106 can act as a type of radial spring for the sheath 104. It can be integrated with the sheath body 82 under preload and/or otherwise be placed to provide a force-tuned setting of outward flex of the sheath when advancing over funnel section 30.

As referenced, the stent body may be made from laser-cut hypotube. Another option is a woven or braided construct made from wire. An example of such a woven stent is provided in U.S. Pat. No. 6,792,979, incorporated herein by reference. Other construction options are possible as well.

For example, the catheter body may itself be cut in a stent-like pattern. In which case, it may be overlaid by a flexible membrane (not shown), or otherwise sealed with a flexible dip-coating polymer such as TICOFLEX.

Moreover, other cell geometries than the diamond pattern shown are possible. Various rings, switch-backs and spirals may be employed in the pattern either to increase tracking flexibility and/or for tuning force and/or expansion.

Another spring-based approach is shown in FIG. 5F with sheath 108. Here a plurality of leaf spring members 110 are included within the sheath lumen. While 3 members are shown, and 4 implied given the nature of the cross-section view, as few as three and as many as 6 or eight such members may advantageously be used. The ends 112 of such members may be embedded in the catheter wall 82 as indicated by dotted line to secure their position. Such a result may be achieved by securing the pieces within an insert that is then fused with sheath body 82 or otherwise.

With the spring members 110 made of metal (e.g., stainless steel, NiTi or another Titanium alloy), they may slide past metal braid 32 used in constructing the funnel section, even at high force without gouging or other marking. However, the spring members may advantageously be made of PEEK in still offer good hardness, lubricity and reasonable spring force properties. When constructed of PEEK (or another termoplastic), their ends 112 are may be fused in place as illustrated.

In each of these embodiments, the sleeve 86, 90, 94, 100, 104 and 108 is adapted (by various means) to present a varying inner diameter to the funnel section for implant (re)capture. In examples 86 and 90, the varying inner diameter is by virtue of tapered or curved features. In examples 94, 100, 104 and 108 the features may be more “active” in terms of flexibility determining radial adjustment. A rigid-fin version of sheath 100 presents variation in diameter around the circumference, whereas the other embodiments include features for overall or gross diametrical change—by flex, actuation (e.g., as with balloon actuation) and/or otherwise by change in configuration (e.g., as with the balloon or stent).

Methods

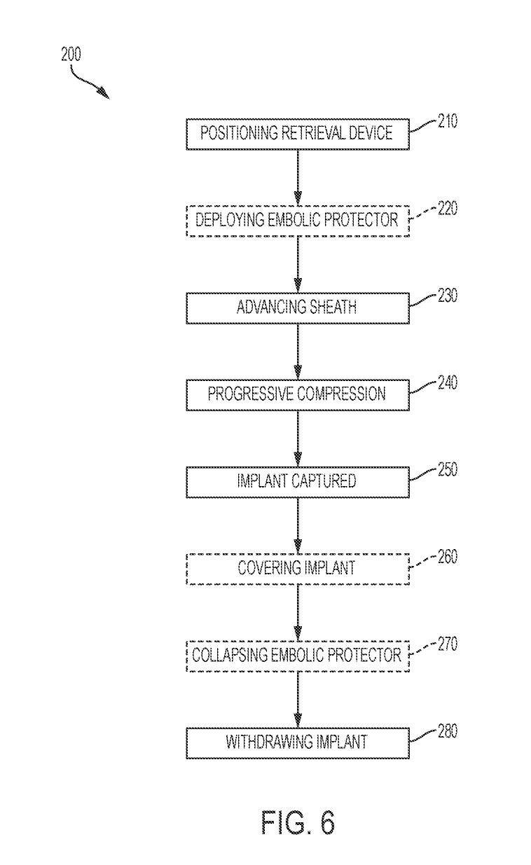

Certain methods have been discussed above. Flowchart 200 in FIG. 6 provides further detail in regard to various options. Specifically, after preparing a retrieval system for use and introducing it into a patient's vasculature as commonly done, a system as described herein is positioned at a target site at 210. The positioning may be adjacent the implant (such as and IVC filter) to be retrieved. In which case, any included embolic protection device may be deployed, at 220, before further advancing or positioning the retriever (note the dashed box indicating optional nature as well as the recursive path in flow chart for further positioning at 210) to place the filter retrieval interface within the funnel trap pocket. Such positioning may include angular manipulation of the device by rotation using a shaped wire, pull-wire apparatus or otherwise.

Once the retrieval device (i.e., the funnel trap portion thereof) is positioned where desired, a sheath (which may be referred to as a locking sheath) is advanced, at 230, over the interface area between the proximal end of the implant and the funnel trap. Either by continued advancement when a tapered or spring-type feature sheath is employed, by internal balloon inflation with such a sheath or by a combination thereof, the interface is progressively compressed at 240. As a result, the implant is securely captured at or by its retrieval interface (i.e., proximal end) at 250.

The method may continue by covering the implant by advancing a/the sheath over its distal legs or any other feature not held within the funnel trap section of the retrieval device. This may be done with the so-called locking sheath, with a separate, secondary (i.e., leg-collapsing) sheath or even with a/the access catheter through which the retrieval system was originally advanced into position.

Alternatively, step or act 260 (and subsequent step or act 270) can be skipped and the implant drawn directly into the sheath and/or access catheter used at 280. However, when the subject system includes an embolic protection device or features, this will typically be closed or collapsed, at 270, prior to implant withdrawal at 280 and completion of the procedure.

Variations

The subject methods, including methods of use and/or manufacture, may be carried out in any order of the events which is logically possible, as well as any recited order of events. Medical methods may include any of a hospital staff's activities associated with device provision, implant positioning, re-positioning, retrieval and/or release.

Furthermore, where a range of values is provided, it is understood that every intervening value, between the upper and lower limit of that range and any other stated or intervening value in the stated range is encompassed within the invention. Also, it is contemplated that any optional feature of the inventive variations described may be set forth and claimed independently, or in combination with any one or more of the features described herein.

Though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention.

Reference to a singular item includes the possibility that there are a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity. Accordingly, the breadth of the different inventive embodiments or aspects described herein is not to be limited to the examples provided and/or the subject specification, but rather only by the scope of the issued claim language.

1. An apparatus for delivery or retrieval of a vascular medical device, the apparatus comprising:
an elongate shaft having an axis having a flexible distal extension comprising braid, the extension folded-back inwardly at a distal opening and forming a more proximal opening, the proximal opening sized to receive and pass an end of the medical device therethrough, and
an elongate sleeve, the sleeve sized for the end of the medical device to be secured in a pocket within the distal extension when the elongate sleeve is advanced over the distal extension,
wherein the sleeve includes a varying inner diameter portion adjacent a distal end.

2. The apparatus of claim 1, wherein the varying inner diameter portion is conical in shape.

3. The apparatus of claim 2, further comprising a distal cylindrical section.

4. The apparatus of claim 3, wherein the distal cylindrical section incorporates a marker band.

5. The apparatus of claim 2, wherein the distal cylindrical section is characterized by an included angle of between about 5 and about 30 degrees.

6. The apparatus of claim 5, wherein the included angle is between about 5 and about 15 degrees.

7. The apparatus of claim 6, wherein the included angle is between about 7 and about 12 degrees.

8. The apparatus of claim 1, wherein the varying inner diameter has a curvilinear profile.

9. The apparatus of claim 1, wherein the profiles are S-shaped.

10. The apparatus of claim 1, wherein the varying inner diameter portion is provided by a balloon within a lumen of the elongate sleeve at a distal end thereof.

11. The apparatus of claim 10, wherein the balloon is toroidal.

12. The apparatus of claim 1, wherein the varying inner diameter is provided by a plurality of internal members, the plurality of internal members being either leaf springs or fins.

13. The apparatus of claim 1, wherein the varying inner diameter portion is provided by flexibility set with a stent-like body.

14. The apparatus of claim 1, further comprising an expandable funnel mounted on the shaft proximal to the extension.

15. The apparatus of claim 1, further comprising an expandable funnel mounted on the elongate sleeve.

16. The apparatus of claim 15, wherein the funnel is mounted proximal to the adjustable inner diameter.

17. A method of vascular medical device retrieval, the method comprising:
positioning a retrieval device comprising a shaft and a folded-back extension defining an interior pocket with the pocket receiving an end of a medical device; and
sliding a sheath over the extension, compressing the extension progressively with a varying diameter of the sheath and capturing the end.

18. The method of claim 17, wherein the progressive compression is by a tapered section.

19. The method of claim 18, wherein the tapered section has a profile selected from conical and curvilinear shapes.

20. The method of claim 17, wherein the progressive compression is by inflation of a balloon within the sheath.
